(19) United States
(12) Patent Application Publication
(10) Pub. No.: US 2022/0099615 A1
(43) Pub. Date: Mar. 31, 2022

(54) Devices, Methods, and Chemical Reagents for Biopolymer Sequencing

(71) Applicant: Universal Sequencing Technology Corporation, Canton, MA (US)

(72) Inventors: Peiming Zhang, Gilbert, AZ (US); Ming Lei, Sharon, MA (US)

(21) Appl. No.: 17/424,141

(22) PCT Filed: Jan. 18, 2020
(86) PCT No.: PCT/US20/14228

§ 371 Date: Jul. 19, 2021

Related U.S. Application Data
(60) Provisional application No. 62/794,096, filed on Jan. 18, 2019.

(51) Int. Cl. G01N 27/327

(52) U.S. Cl. CPC G01N 27/3278

(57) ABSTRACT

This invention provides methods to construct a system for the sequencing of biomolecules based on in vitro template-directed enzymatic replication or synthesis. Embodiments of the present invention are related to systems, methods, devices, and compositions of matter for the sequencing or identification of biopolymers using electronic signals. More specifically, the present disclosure includes embodiments which teach the construction of a system to detect biopolymers electronically based on enzymatic activities, including replication.

### CROSS-REFERENCE TO RELATED APPLICATION

This application claims priority to U.S. Provisional Application Ser. No. 62/794,096 filed Jan. 18, 2019, the entire disclosure of which is hereby incorporated herein by reference.

### FIELD

Embodiments of the present invention are related to systems, methods, devices, and compositions of matter for the sequencing or identification of biopolymers using electronic signals. More specifically, the present disclosure includes embodiments which teach the construction of a system to detect biopolymers electronically based on enzymatic activities, including replication. The biopolymers in the present invention include but not limited to DNA, RNA, DNA oligos, protein, peptides, polysaccharides, etc., either natural or synthesized. The enzymes include but not limited to DNA polymerase, RNA polymerase, DNA helicase, DNA ligase, DNA exonuclease, reverse transcriptase, RNA primase, ribosome, sucrase, lactase, etc., either natural, mutated or synthesized. In the following, mainly DNA and DNA polymerase are discussed and used to illustrate the inventive concept.

### BACKGROUND OF THE INVENTION

DNA sequencing by enzymatic synthesis can be traced back to Sanger's chain termination method, by which dideoxynucleotides are selectively incorporated into DNA by DNA polymerase during in vitro replication of the target sequences.^{1,2} This enzymatic approach has been extended to next-generation sequencing (NGS) in a high throughput or real-time fashion.^{3,4} Although NGS has reduced the cost of sequencing a human genome to a range of $1000, the recent data shows that the cost reduction may have reached a bottom plateau (https://www.genome.gov/27565109/the-cost-of-sequencinq-a-human-genome). One of limiting factors is that NGS relies on fluorescent detection, which requires a sophisticated instrument that is bulky and expensive.

Electrical readout of DNA synthesis by polymerase was stimulated by the label-free detection,⁵ which has been developed as a product that can be used in the genome sequencing.⁶ The recent progress has shown that the electronic approach can be developed as a hand-held device, such as the MinION sequencer (www.nanoporetech.com) that measures changes in ionic currents passing through protein nanopores for DNA sequencing, where a DNA helicase is employed to control the translocation of DNA through the nanopores.⁷ However, the protein nanopore can only achieve a low sequencing accuracy (85% with a single read⁸). Gundlach and coworkers have demonstrated that the ionic current blockage in a protein nanopore composed of Mycobacterium smegmatis porin A (known as MspA) is a collected event of four nucleotides (quadrorner), and therefore there are 4⁴ (Le. 256) possible quadromers that exert a significant number of redundant current levels.^{9,10} Because the ionic current is affected by nucleotides beyond those inside the nanopore,¹¹ the notion of an atomically thin nanopore for sequencing may not be conceivable to achieve a single nucleotide resolution.

Collins and coworkers reported a single wall carbon nanotube (SWCNT) field-effect transistor (FET) device with a Klenow fragment of DNA polymerase I tethered on it to monitor its DNA synthesis.^{12,13} In the device, when a nucleotide was incorporated into a DNA strand, a brief excursion of ΔI(t) below the mean baseline currents was recorded. The incorporation of different nucleotides by the enzyme results in differences in ΔI. This technology can potentially be used in sequencing DNA. The carbon nanotube is a material made from just a single layer of carbon atoms locked in a hexagonal grid. Because of the rigid chemical structure, its sensing may rely on electrostatic gating motions of charged side chains close to the protein attachment site. However, the carbon nanotube in the device had a length of 0.5-1.0 μm,¹⁴ which poses a challenge to mounting a single protein molecule on it reproducibly. In a prior art, the invention (WO 2017/024049) provides a nanoscale field effect transistor (nanoFET) for DNA sequencing, where the DNA polymerase is immobilized with its nucleotide exit region oriented toward a carbon nanotube gate, and it also provides a set of nucleotides with their polyphosphates labeled for the identification of incorporated nucleotides (FIG. 1).

One invention (US 2017/0044605) has claimed an electronic sensor device to sequence DNA and RNA using a polymerase immobilized on a biopolymer that bridges two separate electrodes (FIG. 2). In another prior art (US 2018/0305727, WO 2018/208505), a single enzyme is directly wired to both positive and negative electrodes to complete a circuit such that all electrical currents must flow through the molecule. In addition, the enzyme is attached to electrodes through more than two contacting points. Nonetheless, it requires a sub-10 nm nanogap, which poses a great challenge to manufacture.

In the last decades, programmed self-assembly of nucleic acids (DNA and RNA) has been developed for the construction of nanostructures.^{15, 16} First, the complex DNA nanostructures are constructed based on molecular motifs, such as the Holliday junction,^{17, 18} multi-arm junction,¹⁹ double (DX) and triple crossover (TX) tiles,^{20, 21} paranemic crossover (PX),²² tensegrity triangle,²³ six-helix bundle,²⁴ and single-stranded circular DNA or DNA origami (FIG. 3).²⁵ With these DNA motifs, a size and shape tunable nanostructure can be readily constructed. The DNA nanostructure is more rigid than DNA duplexes and can also be functionalized in a similar way as does the DNA duplex. It provides a unique breadboard for the construction of an electronic biosensor. A 10×60 nm² TX tile was measured to have a conductance of ˜70 pS in a 45-55 nm nanogap under 90% relative humidity.²⁶ Thus, a nanogap bridged by a DNA nanostructure can be employed to construct nanobiodevices for the single molecule detection. It is conceivable that the conductivity of a DNA nanostructure can be tuned by its sequences and structures, structural dynamics. Similarly, RNA nanostructures are constructed using the RNA motifs (FIG. 4) through self-assembling.^{27, 28} RNA is much more versatile in structure and function compared to DNA, and its duplex is thermodynamically more stable than the DNA counterpart. Thus, the RNA nanostructure can be an alternative to the corresponding DNA nanostructure. It has been demonstrated that RNA can mediate the electron transfer as well.²⁹

A recent study has reported that DNA polymerase I bound to a PX motif with a K_{d} of ˜220 nM, and a DX motif with a K_{d} of ˜13 μM in solution.³⁰ Though, the PX motif could not function as a substrate for the polymerase extension. For DNA sequencing, ϕ29 DNA polymerase is an enzyme used in various platforms.^{9, 31, 32} Based on amino acid sequence similarities and its sensitivity to specific inhibitors, the ϕ29 DNA polymerase was included in the eukaryotic-type family B of DNA-dependent DNA polymerases.³³ As any other DNA polymerase, it accomplishes sequential template-directed addition of dNMP units onto the 3′-OH group of a growing DNA chain, showing discrimination for mismatched dNMP insertion by a factor from 10⁴ to 10⁶.³⁴ In addition, ϕ29 DNA polymerase catalyzes 3′-5′ exonucleolysis, i.e. the release of dNMP units from the 3′ end of a DNA strand, degrading preferentially a mismatched primer-terminus, which further enhances the replication fidelity.³⁵⁻³⁷ The ϕ29 DNA polymerase's proofreading activity, strand displacement, and processivity may be attributed to its unique structure (FIG. 5).³⁸⁻⁴⁰

### BRIEF DESCRIPTION OF THE DRAWINGS

FIG. 1: A prior art nanoscale field effect transistor (nanoFET) and an exemplary set of nucleotide analogs carrying differentiable charged conductive labels for DNA sequencing.

FIG. 2: A prior art of using biopolymers to connect a DNA polymerase to electrodes.

FIG. 3: Exemplary DNA motifs for the construction of DNA nanostructures.

FIG. 4: Exemplary RNA motifs for the construction of RNA nanostructures.

FIG. 5: Ribbon representation of the domain organization of φ29.

FIG. 6: A schematic diagram of a single molecule DNA sequencing device.

FIG. 7: Kinetic mechanism of nucleotide binding and incorporation accompanied by conformation changes of the DNA polymerase.

FIG. 8: An illustration on a process of fabricating a nanogap with a passivated substrate, passivated nanowires, and exposed silicon oxide surface in the nanogap area.

FIG. 9: Chemical structures of 5′-mercapto-nucleosides used at the end of DNA nanostructures for attachment to metal electrodes.

FIG. 10: Chemical structures of base chalcogenated nucleosides.

FIG. 11: (a) a tripod containing a carboxyl function as an anchor for attaching DNA nanostructures to metal electrodes; (b) Chemical structures of nucleosides containing an amino function at their respective nucleobases.

FIG. 12: Chemical structures of nucleobase chalcogenated nucleosides.

FIG. 13: Chemical structures of nucleobase chalcogenated nucleosides.

FIG. 14: Electrochemical functionalization of an electrode (cathode) of the nanogap using an N-heterocyclic carbene.

FIG. 15: A schematic diagram of immobilizing a DNA tile on a streptavidin in a nanogap for its attachment to electrodes.

FIG. 16: (a) Chemical structure of a four-arm linker containing two biotins and two silatrane functions; (b) its 3D structure from a molecular mechanics calculation.

FIG. 17: Chemical structures of biotinylated nucleosides.

FIG. 18: A mutant of phi29 DNA polymerase containing p-azidophenylalanine at the locations of 277 and 479 with two tags at its two termini as well as a mutant containing p-azidophenylalanine at the sites 277 and 479. The native structure is adopted from protein data bank (PDB ID: 1XHX).³⁸

FIG. 19: A process of attaching peptides to the termini of phi29 DNA polymerase.

FIG. 20: A crystal structure of Phi29 DNA polymerase complexed with primer-template DNA and incoming nucleotide substrates (PDB ID: 2PYL).

FIG. 21: Chemical structures of nucleosides containing acetylene.

FIG. 22: Chemical structures of nucleoside hexa-phosphates tagged with DNA intercalators.

FIG. 23: A schematic diagram of a single molecule device for direct RNA sequencing.

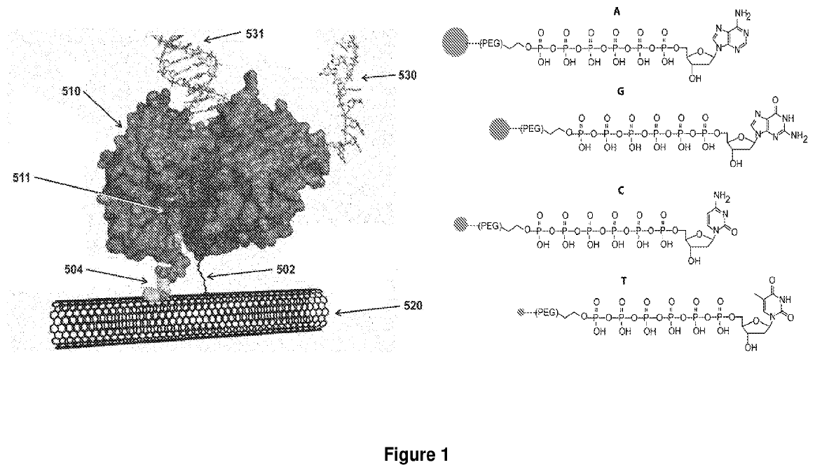

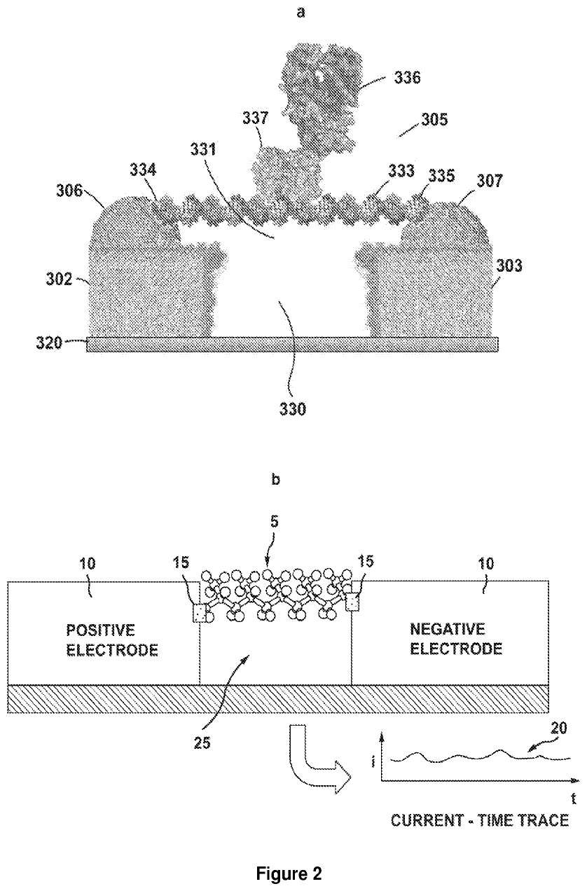

### SUMMARY OF THE INVENTION

This invention provides a device for single-molecule DNA sequencing. As shown in FIG. 6, a 10 nm nanogap is fabricated by semiconductor technology between two electrodes with its surrounds passivated with inert chemicals for the prevention of non-specific adsorption and the inner area of the nanogap exposed for the chemical reactions. A

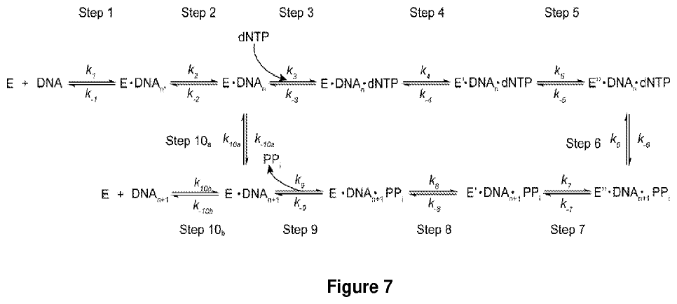

DNA tile is anchored to the electrodes to bridge the nanogap, on which a DNA polymerase, e.g., ϕ29 DNA polymerase, is immobilized. For sequencing, a target DNA is subjected to replication in the device. During the replicating process, nucleotides are incorporated into an elongating DNA strand by the DNA polymerase. Mechanistically, the nucleotide incorporation is accompanied by conformation changes of the polymerase (FIG. 7).⁴¹ Since the polymerase is directly attached to the DNA tile, the conformation change would disturb the tile's structure, resulting in fluctuation of electrical currents that can be used as signatures to identify the incorporation of different nucleotides.

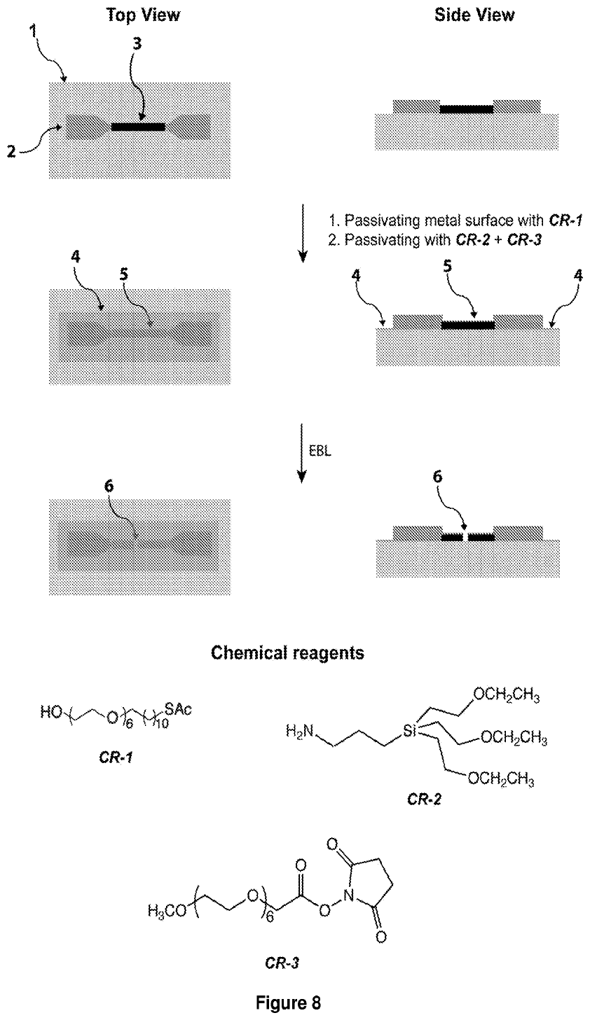

In one embodiment, the invention provides a method to fabricate a nanogap between two electrodes with a size ranging from 3 nm to 1000 nm, preferably from 5 nm to 100 nm, and more preferably from 10 nm to 50 nm. First, electron-beam lithography (EBL) is used to generate metal (such as Au, Pd, and Pt) nanowires. For example, as shown in FIG. 8, a gold nanowire (3) with a dimension of 1000×10×10 nm (Length×Width×Height) is fabricated on a silicon oxide substrate (1) by EBL and connected to the large metal contact pads (2) by standard photolithography techniques. The length of the nanowire is between 100 nm to 100 μm, preferably 1 μm to 10 μm; the width is between 5 nm to 100 nm, preferably 10 nm to 50 nm; and the height (thickness) is between 3 nm to 100 nm, preferably 5 nm to 20 nm. An array of nanowires can also be fabricated by nanoimprinting.⁴² Subsequently, the metal surface is passivated by reacting with 11-mercaptoundecyl-hexaethylene glycol (CR-1)⁴³ to form a monolayer, and the silicon oxide surface is treated first with aminopropyltriethoxysaline (CR-2), followed by reacting with N-hydroxysuccinimidyl 2-(ω-O-methoxy-hexaethylene glycol)acetate (CR-3). At last, the passivated nanowire is cut to generate a 20 nm nanogap by helium focused ion beam milling (He-FIB)⁴⁴ and expose the silicon oxide and the side walls of the electrodes in the cut area.

In some of the embodiments, DNA nanostructures are used to bridge the nanogap. As shown in FIG. 7, a 10 nm nanogap is bridged by a two dimensional DNA nanostructure that is composed of four DNA strands.⁴⁵ There are many methods to form DNA nanostructures with different shapes and sizes in solution through self-assembling.⁴⁶⁻⁴⁸

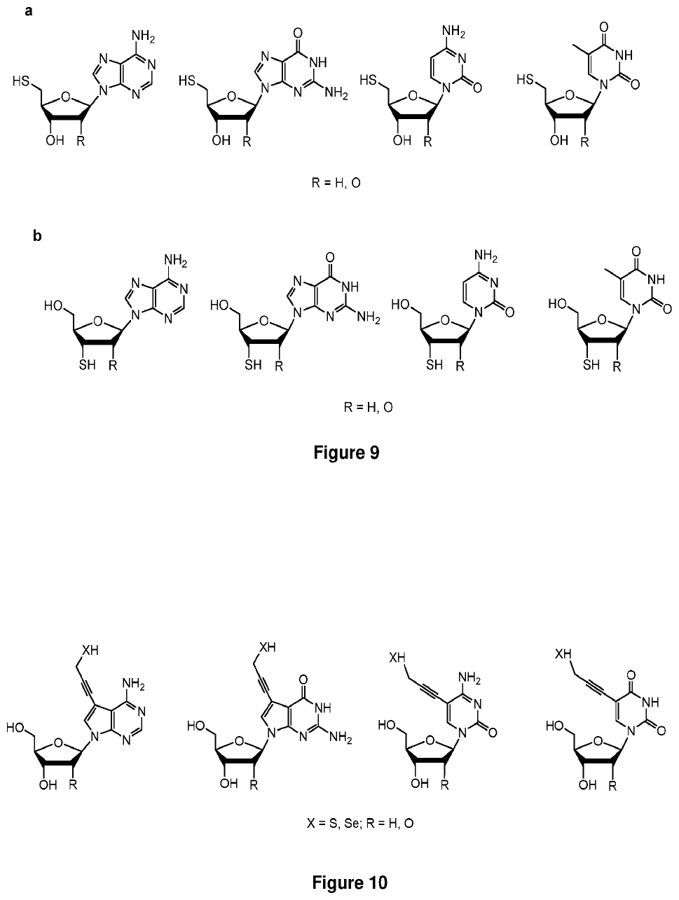

This invention provides methods to attach the said DNA nanostructure to electrodes. In one embodiment, DNA nanostructures are made at their 5′ ends containing 5′-mercaptonucleosides and at their 3′ ends containing 3′-mercaptonucleosides, as shown in FIG. 9. The nucleosides are deoxyribonucleosides (R═H) and ribonucleosides (R═O). Furthermore, the sulfur atom can be replaced by selenium which may be a better anchor for the electron transport.⁴⁹

In another embodiment, the invention provides methods to functionalize the DNA nanostructures at their ends with RXH and RXXR, where R is an aliphatic or aromatic group; X is chalcogens preferring to S and Se.

In some embodiments, the invention provides base chalcogenated nucleosides that can be incorporated into DNA nanostructures for the attachment to electrodes (FIG. 10). It has been demonstrated that connecting the electrodes DNA to electrodes via a nucleobase provides more efficient electrical contact than via the sugar moiety.⁵⁰

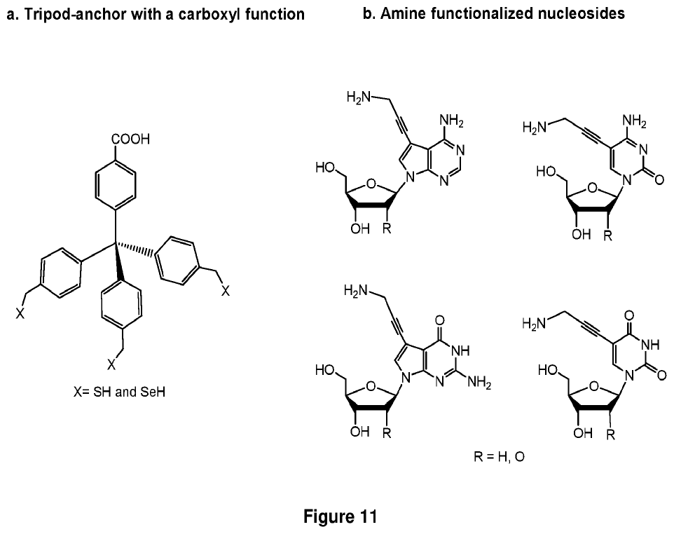

In one embodiment, the invention provides a tripod anchor bearing a tetraphenylmethane with either sulfur (S) or selenium (Se) as an anchoring atom to metal electrodes and the carboxyl group of the tripod for the attachment of a DNA nanostructure (FIG. 11, a). Meanwhile, the DNA nanostructure is modified at their ends with amino functionalized nucleosides (FIG. 11, b) for attachment to the tripod.

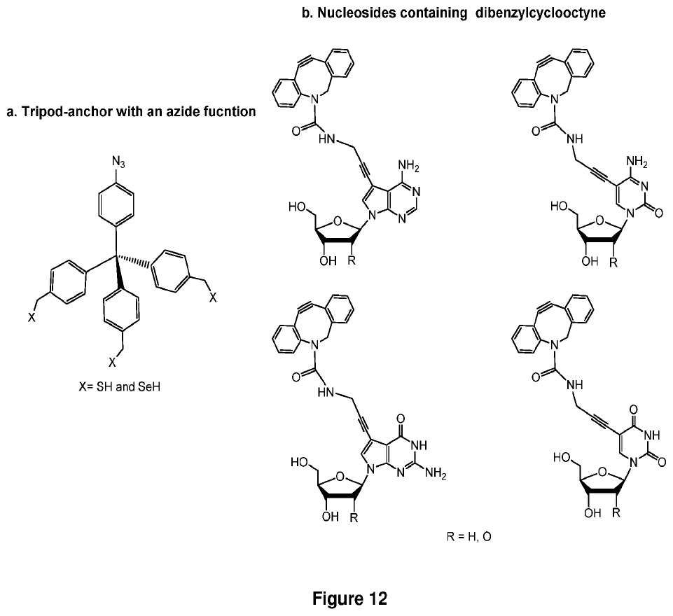

The invention also provides another tripod functionalized with azide (FIG. 12, a), which allows to attachment DNA nanostructures to metal electrodes through the azide-alkyne click reactions. Therefore, the invention provides nucleosides functionalized with cyclooctyne (FIG. 12, b) for the modification of DNA nanostructures at their ends.

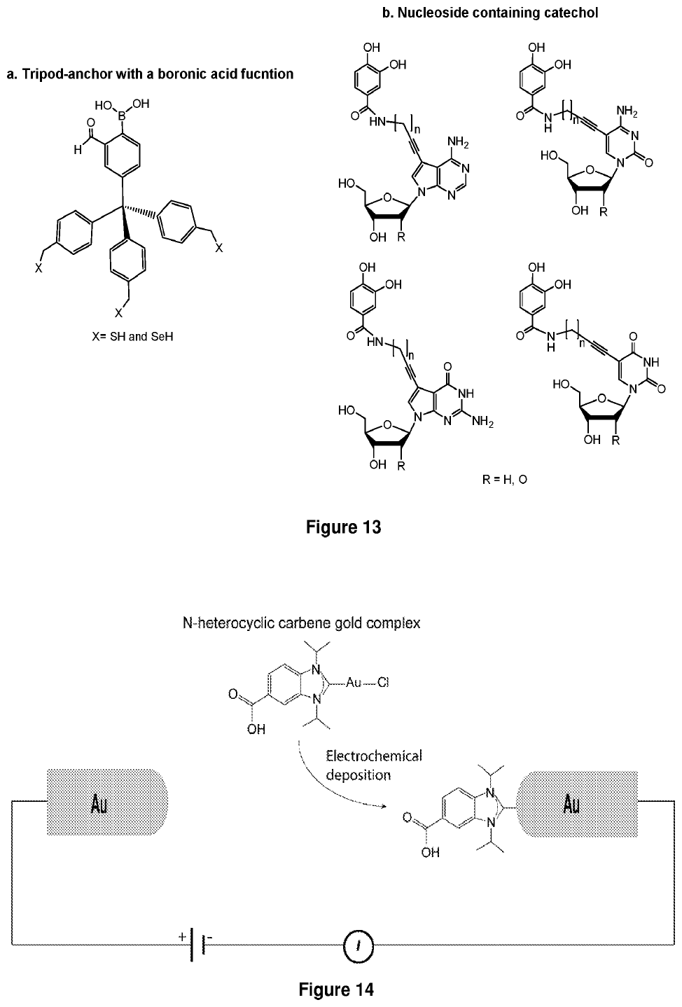

The invention also provides a tripod functionalized with boronic acid (FIG. 13, a) and nucleosides functionalized with diols (FIG. 13, b) for the modification of DNA nanostructures at their ends. Thus, a DNA nanostructure is attached to metal electrodes through the reaction of boronic acid with a diol as disclosed in the previous disclosure (Provisional patent U.S. 62/772,837).

In one embodiment, the invention provides a method to selectively functionalize one of two electrodes with N-heterocyclic carbene (NHC) in a nanogap. As shown in FIG. 14, 5-carboxy-1,3-diisopropyl-1H-benzo[d]imidazol-2-carbene is deposited to a gold electrode by electrochemical reduction of its gold complex in solution.⁵¹ The carboxyl group of the NHC on the electrode is used as an anchor point for attachment by converting it to an activated ester. Thus, a DNA nanostructure bridges a nanogap by its amine functionalized end to react with the NHC electrode and its thiol functionalized end to directly react with the bare gold electrode.

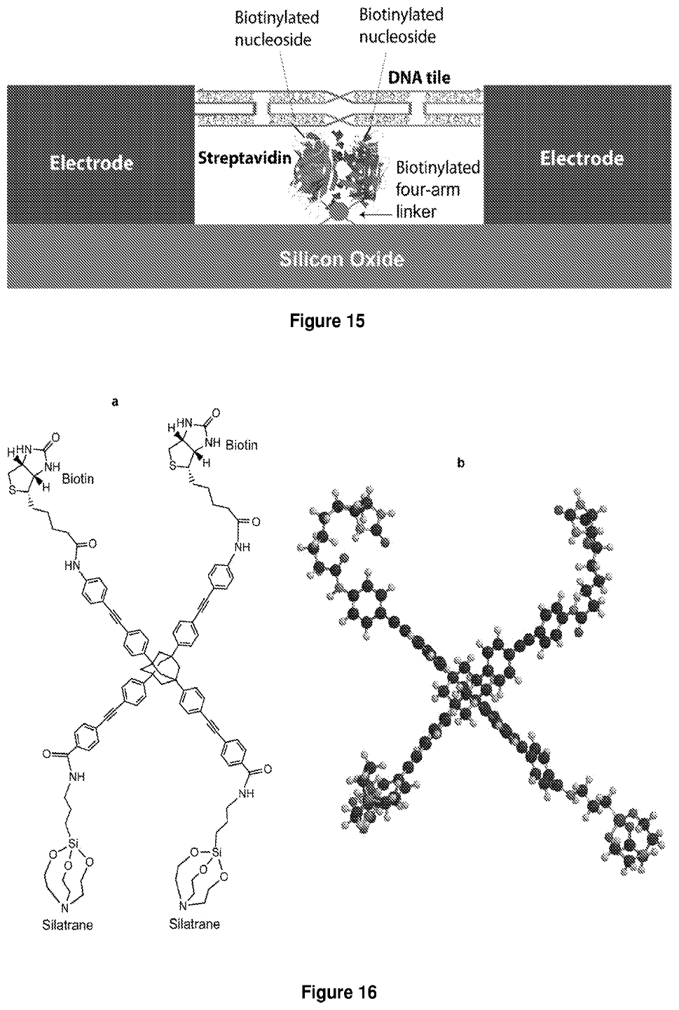

In one embodiment, the invention provides a method to control the location of a nanostructure along the side walls of the electrodes. As illustrated in FIG. 15, a single streptavidin molecule is immobilized in the nanogap through a biotinylated four arm linker so that a biotinylated DNA tile can be connected to the streptavidin, and then attached to the electrodes by one of the methods described above. The invention also provides a four-arm linker, two arms of which are functionalized with biotins and the other two with silatranes (FIG. 16, a), for the streptavidin immobilization. The four-arm linker appears to be a tetrahedron geometry by the molecular mechanics calculation (FIG. 16, b). The two biotin moieties interact with streptavidin to form a bivalent complex. For the streptavidin immobilization, the silatrane moieties first react with silicon oxide, allowing the four-arm linker to be fixed on the surface, followed by the addition of streptavidin to the surface.

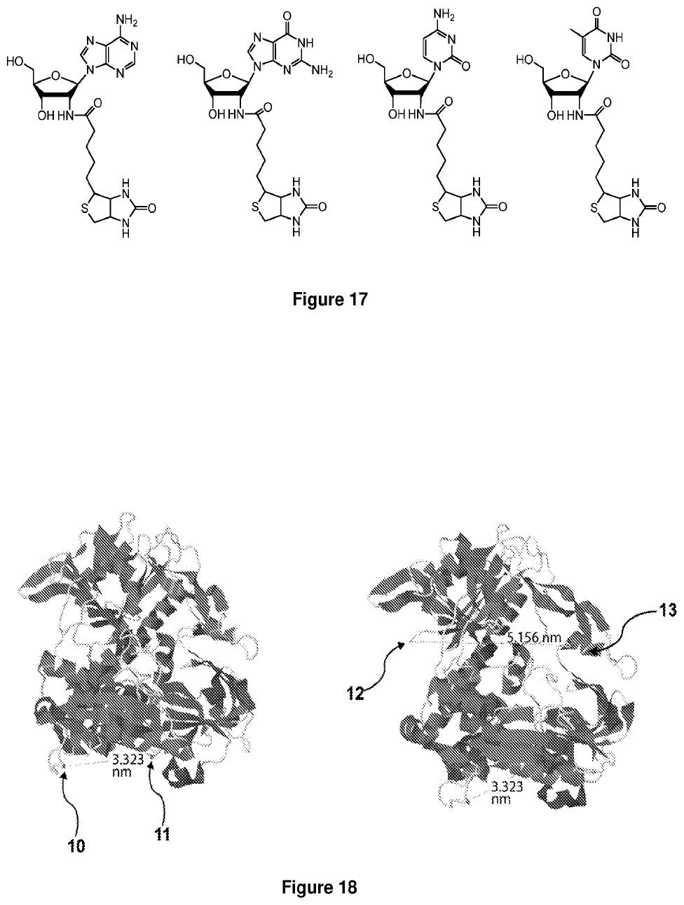

The invention provides biotinylated nucleosides that can be incorporated into DNA through the phosphoramidite chemistry for the construction of DNA nanostructures (FIG. 17).

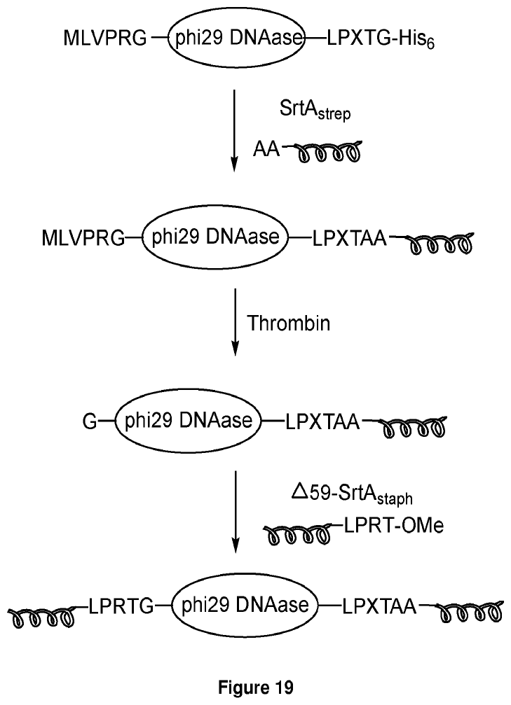

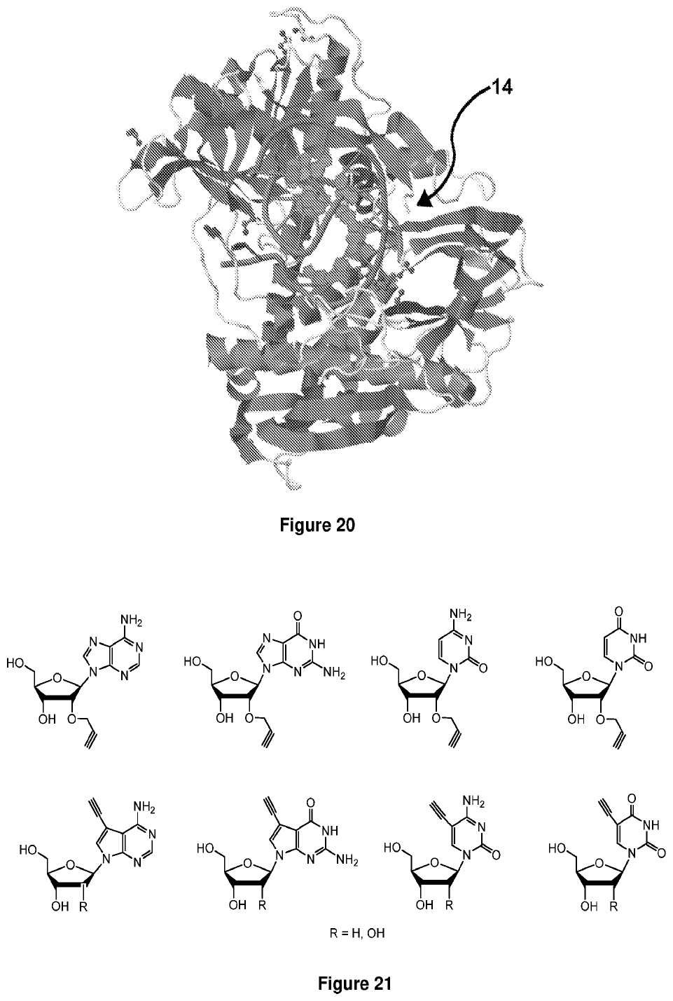

In some embodiments, the invention provides methods to attach a DNA polymerase to the DNA nanostructure. The invention employs both multi-site-directed mutagenesis method⁵² and the genetic code expansion technique⁵³ to substitute unnatural amino acids (UAAs) for canonical amino acids of the DNA polymerase at multiple specific sites. As shown in FIG. 18, a phi29 DNA polymerase mutant is expressed with p-azidophenylalanine substituting for W277 (10) and K479 (11). The UAA p-azidophenylalanine is used for the polymerase immobilization by the click reaction and an aaRS has already been evolved to facilitate its incorporation.^{53,54} The phi29 DNA polymerase mutants are further expressed to have a peptide sequence of MLVPRG at the N terminus (12) and LPXTG-His₆ at the C-terminus (13). In this way, an enzyme can be modified with peptides at its two termini. FIG. 19 shows a process of attaching peptides to the enzyme using Sortases A.⁵⁵ By viewing the structure of Phi29 DNA polymerase complexed with primer-template DNA and incoming nucleotide substrates, we can see the C-terminus (14) of the protein is very close to the DNA (FIG. 20), suggesting that any movement of DNA in the protein could cause a domino effect on the DNA nanostructure, resulting in the fluctuations of electrical currents, which can be used as signatures of the DNA nucleotides incorporating events. Thus, fine tuning the DNA nanostructure can achieve single base resolution.

In one embodiment, the invention provides nucleosides containing acetylene that can be incorporated into DNA for the construction of DNA nanostructures for attaching the DNA polymerase through the click reaction in the presence of a copper catalyst (FIG. 21).

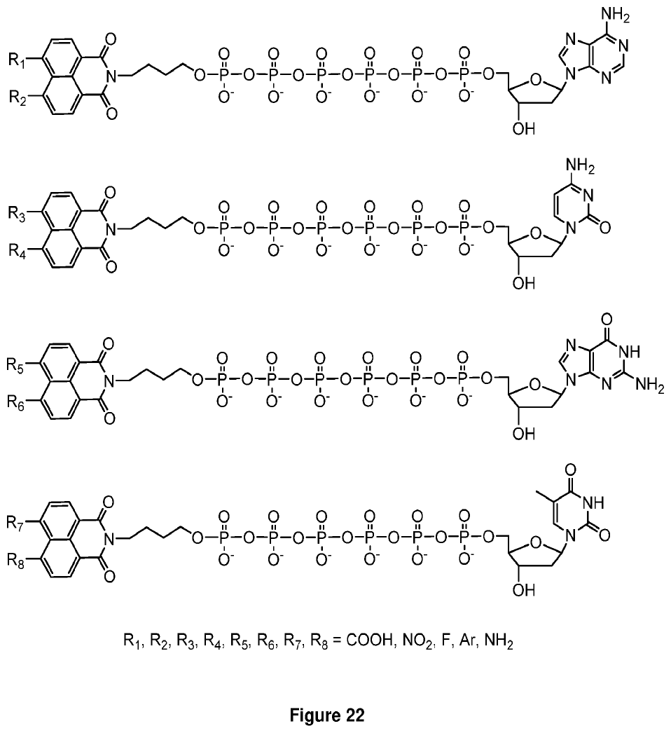

In one embodiment, the invention provides modified nucleotides (dN6P) tagged with different DNA intercalators that interact with DNA nanostructures (FIG. 22). These modified nucleotides are used as substrates for a DNA polymerase to incorporate DNA nucleotides into DNA. First, the DNA polymerase forms a complex with DNA and a nucleoside polyphosphate, which also stabilizes the interaction of the intercalator tag with the DNA nanostructure. When the nucleotide is incorporated into DNA, it releases a pentaphosphate tagged with an intercalator. Because the electrostatic repulsion destabilizes the interaction of intercalator with DNA, it results in the release of the tagged pentaphosphate into solution. Such a process would change the conductance of the DNA nanostructure. Since each dN6P carries a different intercalator, the incorporation of a different nucleotide would cause different current fluctuations, which can be used to identify the nucleotide incorporated into DNA.

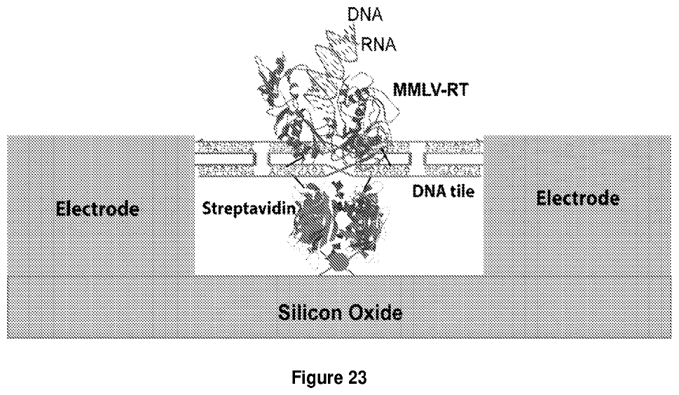

In one embodiment, the invention provides a device for direct sequencing of RNA. As shown in FIG. 23, a reengineered Moloney Murine Leukemia Virus Reverse Transcriptase (M-MLV RT) is irnmobilized on the DNA tile for the RNA reverse transcription. When an RNA target primed with poly(dT) is introduced into the device, DNA nucleotides are incorporated into the poly(dT) primer. In this process, each incorporation causes changes of the polymerase's conformation, resulting in fluctuations of electrical currents. With the incorporating continuation, a train of electric signals is recorded, from which the RNA sequence is deduced with an analytical program.

More specifically, this invention includes the following claimable items (as examples):

1. A system for direct electrical identification and sequencing of a biopolymer in a nanogap comprising a first electrode and the second electrode in proximity to said first electrode, which are bridged by a nucleic acid nanostructure by bonding to both electrodes through chemical bonds that do not break over the time course of a measurement process. An enzyme attached to the nanostructure for carrying out biochemical reactions.

2. Under a bias applied between the first and second electrodes, the device records current fluctuations resulting from the nucleic acid nanostructure's distortions caused by the conformation changes of the enzyme attached to the nanostructure while carrying out biochemical reactions. A bias is chosen between the two electrodes so that a steady DC current is observed, and current fluctuations arise when biochemical reactions take place between the said electrodes. In a polymerization reaction, a train of electrical spikes is recorded for the determination of the polymeric sequences.

3. The said electrodes in claimable item 1 are composed of:
a) metal electrodes that can be functionalized on their surfaces by self-assembling monolayers that can react with anchoring molecules by forming covalent bonds.
b) metal oxide electrodes that can be functionalized with silanes that can react with anchoring molecules to form covalent bonds.
c) carbon electrodes that can be functionalized with organic reagents that can react with anchoring molecules to form covalent bonds.

4. The said nanogap in claimable item 1:
(a) has a length of 3 to 1000 nm, preferably 5 nm to 500 nm, a width of 3 to 1000 nm, preferably 10 to 100 nm, and a depth of 2 to 1000 nm, preferably 2 to 100 nm.
(b) is fabricated on inorganic substrates including silicon and silicon oxide, and polymer films.

5. The said nuclei acid nanostructure in claimable item 1:
(a) has a two-dimensional geometry including rectangle, square, triangle, circles, with a length that can bridge the said two electrodes.
(b) has a three-dimensional geometry including those composed of a bundle of columns, stacked two-dimensional structure, or folded from origami.
(c) is self-assembled from linear or circular DNA in the solution or the nanogap.
(d) is self-assembled from linear or circular RNA in the solution or the nanogap.
(e) is composed of non-phosphate backbone including those peptide, guanidinium, triazole linkages.
(f) includes those bearing sugar modified nucleosides, nucleobase modified nucleosides, nucleoside analogous.
(g) contains functional groups for its attachment to electrodes
(h) contains functional groups for the immobilization of enzymes.

6. The said functional groups for attachment in claimable item 5 are
(a) those thiols on the sugar rings of nucleosides.
(b) those thiols and selenols on the nucleobases of nucleosides.
(c) those aliphatic amines on nucleosides.
(d) Those catechols on nucleoside.

7. The said anchoring molecules in claimable item 3 are
(a) those that can interact with the metal surface through multivalent bonds.
(b) a tripod structure that can interact with the metal surface through trivalent bonds.
(c) Those that are composed of a tetraphenylmethane core of which three phenyl rings are functionalized with —CH₂SH and —CH₂SeH and the last phenyl ring is functionalized with azide, carboxylic acid, boronic acid, and organic groups that can react with those functional groups incorporated into DNA and RNA nanostructures.

8. The said functional groups incorporated into DNA and RNA nanostructures in claimable item 7 are:
(a) Amine functionalized nucleosides that can be incorporated into DNA and RNA by chemical synthesis.
(b) Cyclooctyne and its derivatives functionalized nucleosides that can be incorporated into DNA and RNA by chemical synthesis.
(c) Catechol functionalized nucleosides that can be incorporated into DNA and RNA by chemical synthesis.

9. The said anchoring molecules in claimable 3 are
(a) N-heterocyclic carbenes (NHC);
(b) N-heterocyclic carbenes (NHC) that are selectively deposited to cathode electrodes by electrochemical methods with their metal complexes in solutions.
(c) N-heterocyclic carbenes (NHC) that are deposited to both metal electrodes in organic and aqueous solutions.
(d) N-heterocyclic carbenes (NHC) containing functional groups including amines, carboxylic acids, thiol, boronic acids, or other organic groups for attachment.

10. The said NHC metal complexes in claimable item 8 include those composed of Au, Pd, Pt, Cu, Ag, Ti, TiN, or other transition metals.

11. The said nanogap in claimable item 4 is functionalized with chemical reagents on its bottom.

12. The said chemical reagent in claimable item 11 is:
(a) Silanes that can react with oxide surfaces;
(b) Silatranes that can react with oxide surfaces;
(c) A multi-arm linker that contains silatranes and functional groups;
(d) A four-arm linker that is composed of an adamantane core;
(e) A four-arm linker that contains two silatranes and two biotin moieties.
(f) A four-arm linker that is composed of adamantane core and silatranes and biotin

13. The said chemical reagents in claimable item 12 are used to immobilize proteins in the nanogap, which include antibodies, receptors, streptavidin, avidin.

14. The said streptavidin in claimable item 13 is used to immobilized DNA nanostructures.

15. The said DNA and RNA nanostructures in claimable item 14 is functionalized with biotins by incorporating biotinylated nucleosides into DNA and RNA.

16. The said enzyme in claimable item 1 is recombinant DNA polymerases that carry orthogonal functional groups for their attachment to DNA and RNA nanostructures.

17. The said recombinant DNA polymerases in claimable item 16 are
(a) Those having organic groups at their N- and C-terminals for click reactions on the DNA nanostructures;
(b) Those having unnatural amino acids in their peptide chains for click reactions on the DNA nanostructures;
(c) Those having azide groups at their N- and C-terminals for click reactions on the DNA nanostructures;
(d) Those having 2-amino-6-azidohexanoic acid (6-azido-L-lysine) in their peptide chains for click reactions on the DNA and RNA nanostructures.

18. The said DNA and RNA nanostructure in claimable item 17 are
(a) those containing nucleosides with either their sugar rings or nucleobases functionalized with organic groups for the click reaction;
(b) those containing nucleosides with either their sugar rings or nucleobases functionalized with acetylene groups for the click reaction;

19. The said enzyme in claimable item 1 is recombinant reverse transcriptases that carry orthogonal functional groups for their attachment to DNA and RNA nanostructures.

20. The said recombinant reverse transcriptases in claimable item 19 are
(a) Those having organic groups at their N- and C-terminals for click reactions on the DNA nanostructures;
(b) Those having unnatural amino acids in their peptide chains for click reactions on the DNA nanostructures;
(c) Those having azide groups at their N- and C-terminals for click reactions on the DNA nanostructures;
(d) Those having 2-amino-6-azidahexanoic acid (6-azido-L-lysine) in their peptide chains for click reactions on the DNA and RNA nanostructures.

1. The biochemical reactions in claimable item 1 are
(a) those catalyzed by DNA polymerases using DNA as templates and DNA nucleotides as substrates.
(b) those catalyzed by reverse transcriptases using RNA as templates and DNA nucleotides as substrates.

22. The said DNA nucleotides in claimable item 21 are
(a) DAN nucleoside polyphosphates;
(b) DAN nucleoside polyphosphates tagged with small organic molecules;
(c) DAN nucleoside polyphosphates tagged with intercalators;
(d) DAN nucleoside polyphosphates tagged with minor groove binder;
(e) DAN nucleoside polyphosphates tagged with small drug molecules;

23. The biopolymer in claimable item 1 is selected among the group of DNA, RNA, DNA oligos, protein, peptides, polysaccharides, etc., either natural or synthesized.

24. The enzyme in claimable item 1 is selected among the group of DNA polymerase, RNA polymerase, DNA helicase, DNA ligase, DNA exonuclease, reverse transcriptase, RNA primase, ribosome, sucrase, lactase, etc., and a combination thereof, either natural, mutated or synthesized.

25. The DNA polymerase in claimable item 24 is selected among the group of T7 DNA polymerase, Tag polymerase, DNA polymerase Y, DNA Polymerase Pol I, Pol II, Pol III, Pol IV and Pol V, Pol α (alpha), Pol β (beta), Pol σ (sigma), Pol λ (lambda), Pol δ (delta), Pol ϵ (epsilon), Pol μ (mu), Pol ι (iota), Pol κ (kappa), pol η (eta), terminal deoxynucleotidyl transferase, telomerase, etc., either natural, mutated or synthesized

26. The DNA polymerase in claimable item 24 is Phi29 (□29) DNA polymerase, either natural, mutated or synthesized.

27. The system of claimable item 1 can contain a single nanogap or a plurality of nanogaps, each with a pair of electrodes, an enzyme, a nanostructure and all other features associated with a single nanogap. Furthermore, the system can consist of an array of nanogaps between 100 to 100 million, preferably between 10,000 to 1 million.

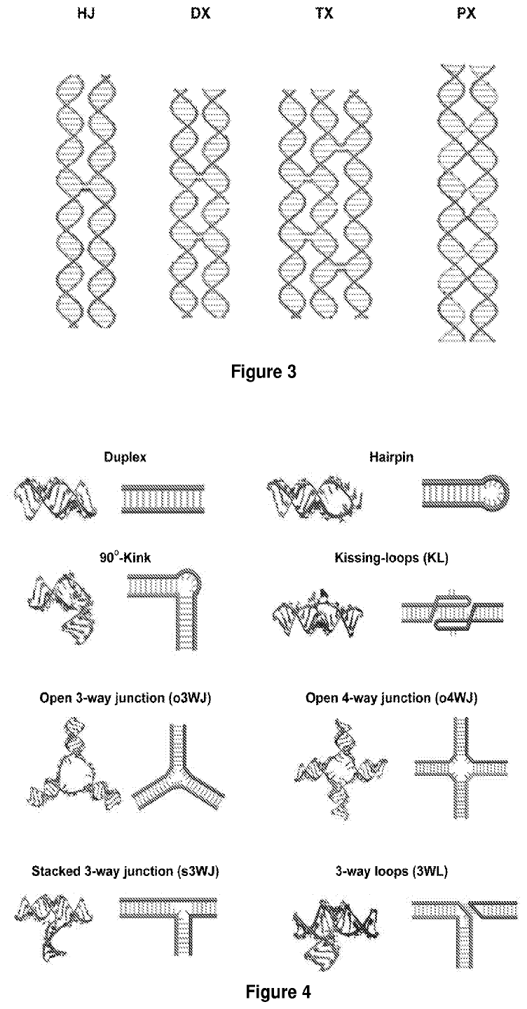

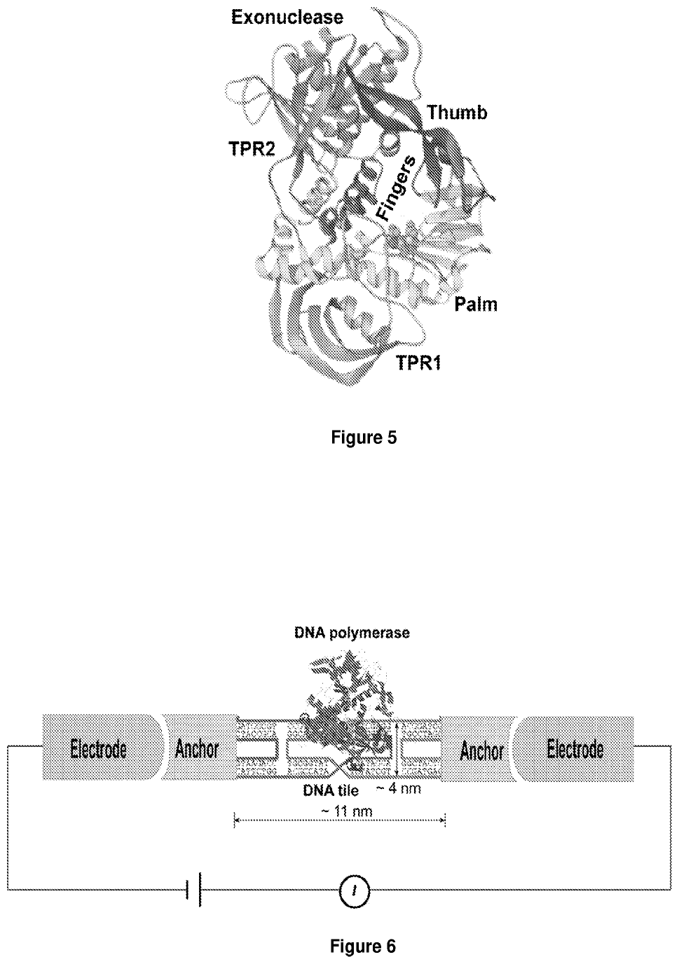

28. The nucleic acid nanostructure in the system of claimable item 1 is selected from the group illustrated in FIGS. 3 and 4.

29. The nucleic acid nanostructure in the system of claimable item 1 can be replaced by other types of nanostructures, such as nanostructures constructed using any organic superconductors by the methods described in the book “Organic Superconductors” by Takehiko Ishiguro⁵⁵.

### General Remarks:

All publications, patents and other documents mentioned herein are incorporated by reference in their entirety.

Unless defined otherwise, all technical and scientific terms used herein have the same meaning as those commonly understood by one of ordinary skill in the art to which this invention belongs. While the present invention has been illustrated by a description of various embodiments and while these embodiments have been described in considerable detail, it is not the intention of the applicants to restrict or in any way limit the scope of the applications. Additional advantages and modifications will be readily appear to those skilled in the art. The invention in its broader aspects is therefore not limited to the specific details, representative device, apparatus and method, and illustrative example shown and described. Accordingly, departures may be made from such details without departing from the spirit of applicant's general inventive concept.

### REFERENCES

1. Sanger, F.; Coulson, A. R., A rapid method for determining sequences in DNA by primed synthesis with DNA polymerase. J. Mol. Biol. 1975, 95, 441-448.

2. Sanger, F.; Nicklen, S.; Coulson, A. R., DNA sequencing with chain-terminating inhibitors. Proc. Nati. Acad. Sci. USA 1977, 74, 5463-5467.

3. Chen, C. Y., DNA polymerases drive DNA sequencing-by-synthesis technologies: both past and present. Front. Microbiol. 2014, 5, 305.

4. Mardis, E. R., DNA sequencing technologies: 2006-2016. Nat. Protoc. 2017, 12, 213-218.

5. Pourmand, N.; Karhanek, M.; Persson, H. H. J.; Webb, C. D.; Lee, T. H.; Zahradni'kova', A.; Davis, R. W., Direct electrical detection of DNA synthesis. Proc. Nati. Acad. Sci. USA 2006, 103, 6466-6470.

6. Rothberg, J. M.; Hinz, W.; Rearick, T. M.; Schultz, J.; Mileski, W.; Davey, M.; Leamon, J. H.; Johnson, K.; Milgrew, M. J.; Edwards, M.; Hoon, J.; Simons, J. F.; Marran, D.; Myers, J. W.; Davidson, J. F.; Branting, A.; Nobile, J. R.; Puc, B. P.; Light, D.; Clark, T. A.; Huber, M.; Branciforte, J. T.; Stoner, I. B.; Cawley, S. E.; Lyons, M.; Fu, Y.; Homer, N.; Sedova, M.; Miao, X.; Reed, B.; Sabina, J.; Feierstein, E.; Schorn, M.; Alanjary, M.; Dimalanta, E.; Dressman, D.; Kasinskas, R.; Sokolsky, T.; Fidanza, J. A.; Namsaraev, E.; McKernan, K. J.; Williams, A.; Roth, G. T.; Bustillo, J., An integrated semiconductor device enabling non-optical genome sequencing. Nature 2011, 475 (7356), 348-52.

7. Cherf, G. M.; Lieberman, K. R.; Rashid, H.; Lam, C. E.; Karplus, K.; Akeson, M., Automated forward and reverse ratcheting of DNA in a nanopore at 5-A precision. Nat. Biotechnol. 2012, 30, 344-8.

8. Jain, M.; Fiddes, I. T.; Miga, K. H.; Olsen, H. E.; Paten, B.; Akeson, M., Improved data analysis for the MinION nanopore sequencer. Nat. methods 2015, 12, 351-6.

9. Manrao, E. A.; Derrington, I. M.; Laszlo, A. H.; Langford, K. W.; Hopper, M. K.; Gillgren, N.; Pavlenok, M.; Niederweis, M.; Gundlach, J. H., Reading DNA at single-nucleotide resolution with a mutant MspA nanopore and phi29 DNA polymerase. Nat. Biotechnol. 2012, 30, 349-353.

10. Laszlo, A. H.; Derrington, I. M.; Ross, B. C.; Brinkerhoff, H.; Adey, A.; Nova, I. C.; Craig, J. M.; Langford, K. W.; Samson, J. M.; Daza, R.; Doering, K.; Shendure, J.; Gundlach, J. H., Decoding long nanopore sequencing reads of natural DNA. Nat. Biotechnol. 2014, 32, 829-833.

11. Lindsay, S., The promises and challenges of solid-state sequencing. Nat Nanotechnol 2016, 11 (2), 109-11.

12. Olsen, T. J.; Choi, Y.; Sims, P. C.; Gul, O. T.; Corso, B. L.; Dong, C.; Brown, W. A.; Collins, P. G.; Weiss, G. A., Electronic measurements of single-molecule processing by DNA polymerase I (Klenow fragment). J. Am. Chem. Soc. 2013, 135, 7855-60.

13. Pugliese, K. M.; Gul, O. T.; Choi, Y.; Olsen, T. J.; Sims, P. C.; Collins, P. G.; Weiss, G. A., Processive Incorporation of Deoxynucleoside Triphosphate Analogs by Single-Molecule DNA Polymerase I (Klenow Fragment) Nanocircuits. J. Am. Chem. Soc. 2015, 137, 9587-94.

14. Choi, Y.; Olsen, T. J.; Sims, P. C.; Moody, I. S.; Corso, B. L.; Dang, M. N.; Weiss, G. A.; Collins, P. G., Dissecting Single-Molecule Signal Transduction in Carbon Nanotube Circuits with Protein Engineering. Nano Lett. 2013,13, 625-631.

15. Xavier, P. L.; Chandrasekaran, A. R., DNA-based construction at the nanoscale: emerging trends and applications. Nanotechnology 2018, 29, 062001.

16. Ge, Z.; Gu, H.; Li, Q.; Fan, C., Concept and Development of Framework Nucleic Acids. J. Am. Chem. Soc. 2018.

17. Sha, R.; Liu, F.; Seeman, N. C., Atomic Force Microscopic Measurement of the Interdomain Angle in Symmetric Holliday Junctions. Biochemistry 2002, 41, 5950-5955.

18. Mao, C.; Sun, W.; Seeman, N. C., Designed Two-Dimensional DNA Holliday Junction Arrays Visualized by Atomic Force Microscopy. J. Am. Chem. Soc. 1999, 121, 5437-5443.

19. Wang, X.; Seeman, N. C., Assembly and Characterization of 8-Arm and 12-Arm DNA Branched Junctions. J. Am. Chem. Soc. 2007, 129, 8169-8176.

20. Fu, T. -J.; Seeman, N. C., DNA Double-Crossover Molecules. Biochemistry & Analytical Biochemistry 1993, 32, 3211-3220.

21. LaBean, T. H.; Yan, H.; Kopatsch, J.; Liu, F.; Winfree, E.; Reif, J. H.; Seeman, N. C., Construction, Analysis, Ligation, and Self-Assembly of DNA Triple Crossover Complexes. J. Am. Chem. Soc. 2000, 122, 1848-1860.

22. Wang, X.; Chandrasekaran, A. R.; Shen, Z.; Ohayon, Y. P.; Wang, T.; Kizer, M. E.; Sha, R.; Mao, C.; Yan, H.; Zhang, X.; Liao, S.; Ding, B.; Chakraborty, B.; Jonoska, N.; Niu, D.; Gu, H.; Chao, J.; Gao, X.; Li, Y.; Ciengshin, T.; Seeman, N. C., Paranemic Crossover DNA: There and Back Again. Chem. Rev. 2018, 10.1021/acs.chemrev.8b00207.

23. Liu, D.; Wang, M.; Deng, Z.; Walulu, R.; Mao, C., Tensegrity: Construction of Rigid DNA Triangles with Flexible Four-Arm DNA Junctions. J. Am. Chem. Soc. 2004, 126, 2324-2325.

24. Mathieu, F.; Liao, S.; Kopatsch, J.; Wang, T.; Mao, C.; Seeman, N. C., Six-Helix Bundles Designed from DNA. Nano Lett. 2005, 5, 661-665.

25. Rothemund, P. W., Folding DNA to create nanoscale shapes and patterns. Nature 2006, 440, 297-302.

26. Linko, V.; Leppiniemi, J.; Paasonen, S. T.; Hytonen, V. P.; Toppari, J. J., Defined-size DNA triple crossover construct for molecular electronics: modification, positioning and conductance properties. Nanotechnology 2011, 22, 275610.

27. Grabow, W. W.; Jaeger, L., RNA self-assembly and RNA nanotechnology. Acc. Chem. Res. 2014, 47, 1871-80.

28. Li, M.; Zheng, M.; Wu, S.; Tian, C.; Liu, D.; Weizmann, Y.; Jiang, W.; Wang, G.; Mao, C., In vivo production of RNA nanostructures via programmed folding of single-stranded RNAs. Nat. Commun. 2018, 9, 2196.

29. Maie, K.; Miyagi, K.; Takada, T.; Nakamura, M.; Yamana, K., RNA-Mediated Electron Transfer: Double Exponential Distance Dependence. J. Am. Chem. Soc. 2009, 131, 13188-13189.

30. Gao, X.; Gethers, M.; Han, S. P.; Goddard, W. A.; Sha, R.; Cunningham, R.; Seeman, N. C., The PX-Motif of DNA Binds Specifically to E. coli DNA Polymerase I. Biochemistry 2018.

31. Korlach, J.; Marks, P. J.; Cicero, R. L.; Gray, J. J.; Murphy, D. L.; Roitman, D. B.; Pham, T. T.; Otto, G. A.; Foquet, M.; Turner, S. W., Selective aluminum passivation for targeted immobilization of single DNA polymerase molecules in zero-mode waveguide nanostructures. Proc. Natl. Acad. Sci. USA 2008, 105, 1176-81.

32. Stranges, P. B.; Palla, M.; Kalachikov, S.; Nivala, J.; Dorwart, M.; Trans, A.; Kumar, S.; Porel, M.; Chien, M.; Tao, C.; Morozova, I.; Li, Z.; Shi, S.; Aberra, A.; Arnold, C.; Yang, A.; Aguirre, A.; Harada, E. T.; Korenblum, D.; Pollard, J.; Bhat, A.; Gremyachinskiy, D.; Bibillo, A.; Chen, R.; Davis, R.; Russo, J. J.; Fuller, C. W.; Roever, S.; Ju, J.; Church, G. M., Design and characterization of a nanopore-coupled polymerase for single-molecule DNA sequencing by synthesis on an electrode array. Proc. Nati. Acad. Sci. USA 2016, 113, E6749-E6756.

33. Bernad, A.; Zabalios, A.; Salas, M.; Blanco, L., Structural and functional relationships between prokaryotic and eukaryotic DNA polymerases. The EMBO journal 1987, 6, 4219-4225.

34. Esteban, J. A.; Salas, M.; Blanco, L., Fidelity of phi29 DNA Polymerase. J. Bio. Chem. 1993, 268, 2713-2726.

35. Blanco, L.; Salas, M., Characterization of a 3′-5′ exonuclease activity in the phage phi29-encoded DNA polymerase. Nucleic Acids Research 1985, 13, 1239-1249.

36. Garmendia, C.; Bernad, A.; Estebane, J. A.; Blanco, L.; Salad, M., The Bacteriophage phi29 DNA Polymerase, a Proofreading Enzyme. J. Bio. Chem. 1992, 267, 2594-2599.

37. Esteban, J. A.; Soengas, M. S.; Salasn, M.; Blanco, L., 3′ to 5′ Exonuclease Active Site of 429 DNA Polymerase. J. Bio. Chem. 1994, 269, 31946-31954.

38. Kamtekar, S.; Berman, A. J.; Wang, J.; Lazaro, J. M.; de Vega, M.; Blanco, L.; Salas, M.; Steitz, T. A., Insights into strand displacement and processivity from the crystal structure of the protein-primed DNA polymerase of bacteriophage phi29. Molecular cell 2004, 16, 609-18.

39. Berman, A. J.; Kamtekar, S.; Goodman, J. L.; Lazaro, J. M.; Vega, M. d.; Blanco, L.; Salas, M.; Steitz, T. A., Structures of phi29 DNA polymerase complexed with substrate: the mechanism of translocation in B-family polymerases. The EMBO journal 2007, 26, 3494-35051.

40. Rodn'guez, I.; zaro, J. M. L.; Blanco, L.; Kamtekar, S.; Berman, A. J.; Wang, J.; Steitz, T. A.; Salas, M.; Vega, M. d., A specific subdomain in □29 DNA polymerase confers both processivity and strand-displacement capacity. Proc. Nati. Acad. Sci. USA 2005, 102, 6407-6412.

41. Raper, A. T.; Reed, A. J.; Suo, Z., Kinetic Mechanism of DNA Polymerases: Contributions of Conformational Dynamics and a Third Divalent Metal Ion. Chem. Rev. 2018, 118, 6000-6025.

42. Pi, S.; Lin, P.; Xia, Q., Fabrication of sub-10 nm metal nanowire arrays with sub-1 nm critical dimension control. Nanotechnology 2016, 27, 464004.

43. Senapati, S.; Biswas, S.; Manna, S.; Ros, R.; Lindsay, S.; Zhang, P., A Y-Shaped Three-Arm Structure for Probing Bivalent Interactions between Protein Receptor-Ligand Using AFM and SPR. Langmuir 2018, 34, 6930-6940.

44. Scholder, O.; Jefimovs, K.; Shorubalko, I.; Hafner, C.; Sennhauser, U.; Bona, G. L., Helium focused ion beam fabricated plasmonic antennas with sub-5 nm gaps. Nanotechnology 2013, 24, 395301.

45. Evans, C. G.; Winfree, E., Physical principles for DNA tile self-assembly. Chem. Soc. Rev. 2017, 46 (12), 3808-3829.

46. Endo, M.; Sugiyama, H., Chemical approaches to DNA nanotechnology. Chembiochem 2009, 10, 2420-43.

47. Hong, F.; Jiang, S.; Lan, X.; Narayanan, R. P.; Sulc, P.; Zhang, F.; Liu, Y.; Yan, H., Layered-Crossover Tiles with Precisely Tunable Angles for 2D and 3D DNA Crystal Engineering. J. Am. Chem. Soc. 2018, 140, 14670-14676.

48. Wang, W.; Lin, T.; Zhang, S.; Bai, T.; Mi, Y.; Wei, B., Self-assembly of fully addressable DNA nanostructures from double crossover tiles. Nucleic Acids Res 2016, 44, 7989-96.

49. Kaull, R. P.; Sawhne, R. S.; Kaul, R.; Engles#, D., Single-Molecule Junctions Based on Selenol Terminated Anchor—A Promising Candidate for Electron Transport. proceedings of the International Conference on Advanced Nanomaterials & Emerging Engineering Technologies 2013, ICANMEET-20J3, 431-435.

50. Xiang, L.; Palma, J. L.; Bruot, C.; Mujica, V.; Ratner, M. A.; Tao, N., Intermediate tunnelling-hopping regime in DNA charge transport. Nat. Chem. 2015, 7, 221-6.

51. Doud, E. A.; Inkpen, M. S.; Lovat, G.; Montes, E.; Paley, D. W.; Steigerwald, M. L.; Vazquez, H.; Venkataraman, L.; Roy, X., In Situ Formation of N-Heterocyclic Carbene-Bound Single-Molecule Junctions. J. Am. Chem. Soc. 2018, 140, 8944-8949.

52. Liang, X.; Peng, L.; Li, K.; Peterson, T.; Katzen, F., A method for multi-site-directed mutagenesis based on homologous recombination. Anal. Biochem. 2012, 427, 99-101.

53. Chatterjee, A.; Sun, S. B.; Furman, J. L.; Xiao, H.; Schultz, P. G., A versatile platform for single- and multiple-unnatural amino acid mutagenesis in Escherichia coli. Biochemistry 2013, 52, 1828-37.

54. Chin, J. W.; Santoro, S. W.; Martin, A. B.; King, D. S.; Wang, L.; Schultz, P. G., Addition of p-Azido-L-phenylalanine to the Genetic Code of Escherichia coli. J. Am. Chem. Soc. 2002, 124, 9026-9027.

55. Antos, J. M.; Chew, G. -L.; Guimaraes, C. P.; Yoder, N. C.; Grotenbreg, G. M.; Popp, M. W. -L.; Ploegh, H. L., Site-Specific N- and C-Terminal Labeling of a Single Polypeptide Using Sortases of Different Specificity. J. Am. Chem. Soc. 2009, 131, 10800-10801.

55. Ishiguro, T., Yamaji, K. and Saito, G., Organic Superconductors, Springer Series in Solid-State Sciences, 2^{nd} Edition, ISSN 0171-1873, 1998.

1. A system for identification, characterization, or sequencing of a biopolymer comprising,
(a) a non-conductive substrate;
(b) a nanogap formed by a first electrode and a second electrode placed next to each other on the non-conductive substrate;
(c) a nanostructure that bridges the said nanogap by attaching one end to the first electrode and another end to the second electrode through chemical bonds, wherein the nanostructure comprises a nucleic acid, either deoxyribonucleic acid (DNA nanostructure) or ribonucleic acid (RNA nanostructure) or a combination thereof;
(d) an enzyme attached to the nanostructure that performs biochemical reactions;
(e) a bias voltage that is applied between the first electrode and the second electrode;
(f) a device that records a current fluctuation through the nanostructure resulting from a distortion within the nanostructure caused by a conformation change initiated by the enzyme attached to the nanostructure; and
(g) a software for data analysis that identifies the biopolymer or a subunit of the biopolymer.

2. The system of claim 1, wherein the non-conductive substrate comprises the following: silicon, silicon oxide, silicon nitride, glass, hafnium dioxide, any metal oxide, any non-conductive polymer film, silicon with silicon oxide or silicon nitride or other non-conductive coating, glass with silicon nitride coating, any non-conductive organic material, and/or any non-conductive inorganic material.

3. The system of claim 1, wherein the biopolymer is selected from the group consisting of DNA, RNA, oligonucleotides, protein, peptides, polysaccharides, either natural, modified or synthesized of any of the aforementioned biopolymers, and a combination thereof.

4. The system of claim 1, wherein the enzyme is selected a from the group consisting of DNA polymerase, RNA polymerase, DNA helicase, DNA ligase, DNA exonuclease, reverse transcriptase, RNA primase, ribosome, sucrase, lactase, either native, mutated, expressed, or synthesized of any of the aforementioned enzymes, and a combination thereof.

5. The system of claim 4, wherein the enzyme is selected from the group consisting of T7 DNA polymerase, Tag polymerase, DNA polymerase Y, DNA Polymerase Pol I, Pol II, Pol III, Pol IV and Pol V, Pol α (alpha), Pol β (beta), Pol σ (sigma), Pol λ (lambda), Pol δ (delta), Pol ϵ (epsilon), Pol μ (mu), Pol ι (iota), Pol κ (kappa), pol η (eta), terminal deoxynucleotidyl transferase, telomerase, either native, mutated, expressed, or synthesized of any of the aforementioned enzymes, and a combination thereof.

6. The system of claim 4, wherein the DNA polymerase is Phi29 (ϕ29) DNA polymerase, either native, mutated, expressed, or synthesized.

7. The system of claim 1, wherein
the two electrodes forming the nanogap are separated by a distance of about 2 nm to about 1000 nm, preferably about 5 nm to about 500 nm, and most preferably about 5 nm to about 50 nm

8. The system of claim 1, wherein
the ends of the electrodes have a substantial rectangular face with a width of about 3 nm to about 1000 nm, preferably about 10 to about 100 nm, and a depth of about 2 nm to about 1000 nm, preferably about 2 nm to about 100 nm.

9. The system of claim 1, wherein the said electrodes are comprised of:
d) metal electrodes that can react with thiol, amine, selenol, and other organic functions;
e) metal electrodes that can be functionalized on the surface by self-assembling monolayers that can react with an anchoring molecule to form covalent bonds;
f) metal oxide electrodes that can be functionalized with silanes that can react with the anchoring molecule to form covalent bonds; or
g) carbon electrodes that can be functionalized with organic reagents that can react with the anchoring molecule to form covalent bonds.

10. The system of claim 1, wherein the electrodes and the substrate are covered by an insulation layer except the end surfaces of the electrodes at the nanogap are not covered.

11. The system of claim 10, wherein the insulation layer comprises a monolayer or multi-layers of passivated inert chemical.

12. The system of claim 11, wherein the inert chemical comprises 11-mercaptoundecyl-hexaethylene glycol (CR-1) for a metal surface passivation, and aminopropyltriethoxysaline (CR-2) & N-hydroxysuccinimidyl 2-(ω-O-methoxy-hexaethylene glycol)acetate (CR-3) for the substrate surface passivation.

13. The system of claim 1, wherein
the nucleic acid nanostructure is self-assembled from either linear and/or circular DNAs; or linear and/or circular RNAs or a combination thereof.

14. The system of claim 1, wherein
the nucleic acid nanostructure has the following shape:
(i) a substantially one-dimensional geometry, such as a linear DNA or a linear RNA structure;
(j) a substantially two-dimensional geometry, including but not limited to, a substantially rectangular structure, a substantially square structure, a substantially triangular structure, a substantially circular structure, or a combination thereof;
(k) a substantially three-dimensional geometry, including but not limited to, a substantially cylindrical structure, a substantially hollow tube structure, a substantially column-like structure, a geometry comprising a substantially bundle-of-columns structure, a geometry comprising a substantially stacked two-dimensional structure, a geometry comprising a substantially folded origami-like structure, or a combination thereof;

15. The system of claim 1, wherein the nanostructure comprises the following:
a. a non-phosphate backbone comprising an amide, a guanidinium, or a triazole linkage;
b. a sugar modified nucleoside or nucleoside analog; and/or
c. a nucleobase with a modified nucleoside or nucleoside analog;

16. The system of claim 1, wherein the nanostructure comprises the following:
a. a functional group configured for attachment to electrodes; and/or
b. a functional group configured for immobilization of the enzyme.

17. The system of claim 16, wherein the functional group configured for electrode attachment comprises
(e) a thiol on a sugar ring of a nucleoside;
(f) a thiol and a selenol on a nucleobase of a nucleoside;
(g) a aliphatic amine on a nucleoside; and/or
(h) a catechol on a nucleoside;
and the functional group configured for immobilization of the enzyme comprises:
(d) an amine functionalized nucleoside that is incorporated into DNA and RNA by chemical or enzymatic synthesis;
(e) a cyclooctyne and/or a derivative functionalized nucleoside that is incorporated into DNA and RNA by chemical or enzymatic synthesis; and/or
(f) a catechol functionalized nucleoside that is incorporated into DNA and RNA by chemical or enzymatic synthesis.

18. The system of claim 9, wherein the anchoring molecule comprises
(d) a molecule configured to interact with a metal surface through multivalent bonds;
(e) a tripod structure configured to interact with a metal surface through trivalent bonds; or
(f) a molecule comprised of a tetraphenylmethane core wherein three of its phenyl rings are functionalized with —CH₂SH and —CH₂SeH and a phenyl ring is functionalized with azide, carboxylic acid, boronic acid, and/or organic groups configured to react with a functional group incorporated into the DNA and/or RNA nanostructure.

19. The system of claim 9, wherein the anchoring molecule comprises
(e) a N-heterocyclic carbene (NHC);
(f) a N-heterocyclic carbene (NHC) in a metal complex configured to be selectively deposited on a cathode electrode by an electrochemical method in solution;
(g) a N-heterocyclic carbene (NHC) configured to be deposited on both metal electrodes in organic and/or aqueous solutions; and/or
(h) a N-heterocyclic carbenes (NHC) containing functional groups comprising an amine, a carboxylic acid, a thiol, a boronic acid, and/or any organic group configured for attachment.

20. The system of claim 19, wherein
the metal complex comprises Au, Pd, Pt, Cu, Ag, Ti, and/or any transition metal.

21. The system of claim 1, further comprising:
a protein configured to be immobilized at the bottom of the nanogap to support and stabilize the nucleic acid nanostructure.

22. The system of claim 21, wherein
the non-conductive bottom of the said nanogap is functionalized with a chemical reagent to immobilize proteins, wherein the chemical reagent comprises:
(g) a silane configured to react with an oxide surface;
(h) a silatrane configured to react with an oxide surface;
(i) a multi-arm linker that comprises a silatrane and a functional group;
(j) a four-arm linker that comprises an adamantane core;
(k) a four-arm linker that comprises two silatranes and two biotin moieties; and/or
(l) a four-arm linker that comprises an adamantane core and a silatrane and a biotin.

23. The system of claim 21, wherein the protein is selected from the group consisting of an antibody, a receptor, an aptamer, and a combination thereof.

24. The system of claim 21, wherein the protein is a streptavidin or an avidin.

25. The system of claim 1, wherein the nanostructure is functionalized with a biotin.

26. The system of claim 1, wherein the enzyme is a recombinant DNA polymerase or a recombinant reverse transcriptase that has an orthogonal functional group configured to attach to the nanostructure.

27. The system of claim 26, wherein the recombinant DNA polymerase comprises
(e) an organic group at an N- and/or C-terminal configured for a click reaction on the DNA nanostructure;
(f) an unnatural, modified or synthetic amino acids configured for a click reaction on the DNA nanostructure;
(g) an azide group at an N- and/or C-terminal configured for a click reaction on the DNA nanostructure; and/or
(h) a 2-amino-6-azidohexanoic acid (6-azido-L-lysine) configured for a click reaction on the DNA and/or RNA nanostructure.

28. The system of claim 27, wherein the nucleic acid nanostructure comprises
(a) a nucleoside with a sugar ring and/or a nucleobase functionalized with an organic group configured for a click reaction;
(b) a nucleoside with a sugar rings or a nucleobase functionalized with an acetylene group configured for a click reaction.

29. The system of claim 26, wherein the recombinant reverse transcriptase comprises
(e) an organic group at an N- and/or C-terminal configured for a click reaction on the DNA nanostructure;
(f) an unnatural, modified, or synthetic amino acid configured for a click reaction on the DNA nanostructure;
(g) an azide group at an N- and/or C-terminal configured for a click reaction on the DNA nanostructure; and/or
(h) a 2-amino-6-azidohexanoic acid (6-azido-L-lysine) configured for a click reaction on the DNA and/or RNA nanostructure.

30. The system of claim 1, wherein the biochemical reaction comprises
(c) a reaction catalyzed by a DNA polymerase using a DNA as a template and a DNA nucleotide as a substrate; and/or
(d) a reaction catalyzed by a reverse transcriptase using a RNA as a template and a DNA nucleotide as a substrate.

31. The system of claim 30, wherein the DNA nucleotide comprises
(a) a DNA nucleoside polyphosphate;
(b) a DNA nucleoside polyphosphate tagged with an organic molecule;
(c) a DNA nucleoside polyphosphate tagged with an intercalator;
(d) a DNA nucleoside polyphosphate tagged with a minor groove binder; and/or
(e) a DNA nucleoside polyphosphate tagged with a drug molecule.

32. The system of claim 1, wherein the nanogap comprises a plurality of nanogaps, each comprising a pair of electrodes, an enzyme, a nanostructure and any feature associated with a single nanogap.

33. The system of claim 32, wherein the plurality of nanogaps form an array of nanogaps between about 100 to about 100 million nanogaps, preferably between about 10,000 to about 1 million nanogaps.

34. The system of claim 1, wherein the nucleic acid nanostructure is selected from the group consisting of a DNA origami-like structure with a Holliday junction (HJ), a multi-arm junction, a double crossover (DX) tile, a triple crossover (TX) tile, a paranemic crossover (PX), a tensegrity triangle, a six-helix bundle, and a single-stranded circular DNA or DNA origami, or a combination thereof, and a DNA tile-like structure with a duplex, a hairpin, a 90°-kink, a kissing-loop, an open 3-way junction, an open 4-way junction, a stacked 3-way junction, or a 3-way loops, or a combination thereof.

35. The system of claim 1, wherein the nucleic acid nanostructure comprises an organic superconductor.

36. A method for identifying, characterizing, or sequencing a biopolymer comprising,
(a) providing a non-conductive substrate;
(b) building a nanogap by placing a first electrode and a second electrode next to each other on the substrate;
(c) providing a nanostructure with a sufficient length to bridge the nanogap, wherein the nanostructure comprises a nucleic acid, either deoxyribonucleic acid (DNA nanostructure) or ribonucleic acid (RNA nanostructure) or a combination thereof;
(d) providing an enzyme that performs a biochemical reaction with the biopolymer;
(e) attaching one end of the nanostructure to the first electrode of the nanogap, and another end to the second electrode wherein the nanogap is bridged, and then attaching the enzyme to the nanostructure; or alternatively, attaching the enzyme to the nanostructure, and then attaching the nanostructure to the nanogap;
(f) providing a bias voltage between the first electrode and the second electrode;
(g) providing a device for recording a current fluctuation through the nanostructure resulting from a distortion within the nanostructure caused by a conformation change initiated by the enzyme attached to the nanostructure; and
(h) providing a data analysis software that is used to identify the biopolymer or a subunit of the biopolymer.

37. The method of claim 36, wherein the non-conductive substrate comprises the following: silicon, silicon oxide, silicon nitride, glass, hafnium dioxide, any metal oxide, any non-conductive polymer film, silicon with silicon oxide or silicon nitride or other non-conductive coating, glass with silicon nitride coating, any non-conductive organic material, and/or any non-conductive inorganic material.

38. The method of claim 36, wherein the biopolymer is selected from the group consisting of DNA, RNA, oligonucleotides, protein, peptides, polysaccharides, either natural, modified or synthesized of any of the aforementioned biopolymers, and a combination thereof.

39. The method of claim 36, wherein the enzyme is selected from the group consisting of DNA polymerase, RNA polymerase, DNA helicase, DNA ligase, DNA exonuclease, reverse transcriptase, RNA primase, ribosome, sucrase, lactase, either native, mutated, expressed, or synthesized of any of the aforementioned enzymes, and a combination thereof.

40. The method of claim 39, wherein the enzyme is selected from the group consisting of T7 DNA polymerase, Tag polymerase, DNA polymerase Y, DNA Polymerase Pol I, Pol II, Pol III, Pol IV and Pol V, Pol α (alpha), Pol β (beta), Pol σ (sigma), Pol λ (lambda), Pol δ (delta), Pol ϵ (epsilon), Pol μ (mu), Pol ι (iota), Pol κ (kappa), pol η (eta), terminal deoxynucleotidyl transferase, telomerase, either native, mutated, expressed, or synthesized.

41. The method of claim 39, wherein the DNA polymerase is Phi29 (ϕ29) DNA polymerase, either native, mutated, expressed, or synthesized.

42. The method of claim 36, wherein
the two electrodes forming the nanogap are separated by a distance of about 2 nm to about 1000 nm, preferably about 5 nm to about 500 nm, and most preferably about 5 nm to about 50 nm

43. The method of claim 36, wherein
the ends of the electrodes have a substantial rectangular face with a width of about 3 nm to about 1000 nm, preferably about 10 nm to about 100 nm, and a depth of about 2 nm to about 1000 nm, preferably about 2 nm to about 100 nm.

44. The method of claim 36, wherein the said electrodes are comprised of:
(a) metal electrodes that can react with thiol, amine, selenol, and other organic functions;
(b) metal electrodes that can be functionalized on the surface by self-assembling monolayers that can react with an anchoring molecules to form covalent bonds;
(c) metal oxide electrodes that can be functionalized with silanes that can react with the anchoring molecules to form covalent bonds; and/or
(d) carbon electrodes that can be functionalized with organic reagents that can react with the anchoring molecules to form covalent bonds.

45. The method of claim 36, wherein
the nucleic acid nanostructure is self-assembled from either linear and/or circular DNAs or linear and/or circular RNAs or a combination thereof.

46. The method of claim 36, wherein
the nucleic acid nanostructure has the following shape:
(a) a substantially one-dimensional geometry, such as a linear DNA or a linear RNA structure;
(b) a substantially two-dimensional geometry, including but not limited to, a substantially rectangular structure, a substantially square structure, a substantially triangular structure, a substantially circular structure, or a combination thereof;
(c) a substantially three-dimensional geometry, including but not limited to, a substantially cylindrical structure, a substantially hollow tube structure, a substantially column-like structure, a geometry comprising a substantially bundle-of-columns structure, a geometry comprising a substantially stacked two-dimensional structure, a geometry comprising a substantially folded origami-like structure, or a combination thereof;

47. The method of claim 36, wherein the nanostructure contains the following:
a. a non-phosphate backbone comprising an amide, an guanidinium, or a triazole linkage;
b. a sugar modified nucleoside or nucleoside analog; and/or
c. a nucleobase with a modified nucleoside or nucleoside analog;

48. The method of claim 36, wherein the nanostructure contains the following:
a. a functional group configured for attachment to electrodes; and/or
b. a functional group configured for immobilization of the enzyme.

49. The method of claim 48, wherein the functional group for electrode attachment comprises
(a) a thiol on a sugar ring of a nucleoside;
(b) a thiols and a selenol on a nucleobase of a nucleoside;
(c) a aliphatic amine on a nucleoside; and/or
(d) a catechol on a nucleoside;
and the functional group for the immobilization of the enzyme includes:
(a) an amine functionalized nucleoside that is incorporated into DNA and RNA by chemical or enzymatic synthesis;
(b) a cyclooctyne and/or a derivative functionalized nucleoside that is incorporated into DNA and RNA by chemical or enzymatic synthesis; and/or
(c) a catechol functionalized nucleoside that is incorporated into DNA and RNA by chemical or enzymatic synthesis.

50. The method of claim 44, wherein the said anchoring molecule includes
(a) a molecule configured to interact with a metal surface through multivalent bonds;
(b) a tripod structure configured to interact with a metal surface through trivalent bonds; or
(c) a molecule comprised of a tetraphenylmethane core wherein three of its phenyl rings are functionalized with —CH₂SH and —CH₂SeH and a phenyl ring is functionalized with azide, carboxylic acid, boronic acid, and/or organic groups configured to react with a functional group incorporated into the DNA and/or RNA nanostructure.

51. The method of claim 44, wherein the said anchoring molecule comprises
(a) a N-heterocyclic carbenes (NHC);
(b) a N-heterocyclic carbene (NHC) in a metal complex configured to be selectively deposited on a cathode electrode by an electrochemical method in solution;
(c) a N-heterocyclic carbene (NHC) configured to be deposited to both metal electrodes in organic and/or aqueous solutions,
(d) a N-heterocyclic carbene (NHC) containing functional groups comprising an amine, a carboxylic acid, a thiol, a boronic acid, and/or any organic group configured for attachment.

52. The method of claim 51, wherein
the metal complex comprises Au, Pd, Pt, Cu, Ag, Ti, and/or any transition metal.

53. The method of claim 36, further comprising
providing a protein configured to be immobilized at the bottom of the nanogap to support and stabilize the nucleic acid nanostructure.

54. The method of claim 53, further comprising
functionalizing the bottom of the nanogap with a chemical reagent to immobilize proteins, wherein the said chemical reagent comprises:
(a) a silane configured to react with an oxide surface;
(b) a silatrane configured to react with an oxide surface;
(c) a multi-arm linker that comprises a silatrane and a functional group;
(d) a four-arm linker that comprises an adamantane core;
(e) a four-arm linker that comprises two silatranes and two biotin moieties; and/or
(f) a four-arm linker that comprises an adamantane core and a silatrane and a biotin.

55. The method of claim 53, wherein the protein is selected from the group consisting of an antibody, a receptor, an aptamer, and a combination thereof.

56. The method of claim 53, wherein the protein is a streptavidin or an avidin.

57. The method of claim 36, wherein the nanostructure is functionalized with a biotin.

58. The method of claim 36, wherein the enzyme is a recombinant DNA polymerase or a recombinant reverse transcriptase that has an orthogonal functional group configured to attach to the nanostructure.

59. The method of claim 58, wherein the said recombinant DNA polymerase comprises
(a) an organic groups at an N- and/or C-terminal configured for a click reaction on the DNA nanostructure;
(b) an unnatural, modified or synthetic amino acid configured for a click reaction on the DNA nanostructure;
(c) an azide group atan N- and/or C-terminal for a click reaction on the DNA nanostructure; or
(d) a 2-amino-6-azidohexanoic acid (6-azido-L-lysine) configured for a click reaction on the DNA and/or RNA nanostructure.

60. The method of claim 59, wherein the nucleic acid nanostructure comprises
(a) a nucleoside with a sugar ring and/or a nucleobase functionalized with an organic group for a click reaction;
(b) a nucleoside with a sugar ring or a nucleobase functionalized with an acetylene group for a click reaction.

61. The method of claim 58, wherein the recombinant reverse transcriptase are
(a) an organic groups at an N- and/or C-terminal configured for a click reaction on the DNA nanostructures;
(b) an unnatural, modified, or synthetic amino acid configured for a click reaction on the DNA nanostructure;
(c) an azide group at an N- and C-terminal configured for a click reaction on the DNA nanostructure; and/or
(d) a 2-amino-6-azidohexanoic acid (6-azido-L-lysine) configured for a click reaction on the DNA and/or RNA nanostructure.

62. The method of claim 36, wherein the biochemical reaction comprises
(e) a reaction catalyzed by a DNA polymerase using a DNA as a template and a DNA nucleotide as a substrate; and/or
(f) a reaction catalyzed by a reverse transcriptase using a RNA as template and a DNA nucleotide as a substrate.

63. The method of claim 62, wherein the DNA nucleotide comprises
(a) a DNA nucleoside polyphosphate;
(b) a DNA nucleoside polyphosphate tagged with an organic molecule;
(c) a DNA nucleoside polyphosphate tagged with an intercalator;
(d) a DNA nucleoside polyphosphate tagged with a minor groove binder; and/or
(e) a DNA nucleoside polyphosphate tagged with a drug molecule;

64. The method of claim 36, wherein the nanogap comprises a plurality of nanogaps, each comprising a pair of electrodes, an enzyme, a nanostructure and any feature associated with a single nanogap.

65. The method of claim 64, wherein the plurality of nanogaps form an array of nanogaps between about 100 to about 100 million, preferably between about 10,000 to about 1 million.

66. The method of claim 36, wherein the nucleic acid nanostructure is selected from the group consisting of a DNA origami-like structures with a Holliday junction (HJ), a multi-arm junction, a double crossover (DX) tile, a triple crossover (TX) tile, a paranemic crossover (PX), a tensegrity triangle, a six-helix bundle, and a single-stranded circular DNA or DNA origami, or a combination thereof, and a DNA tile-like structure with a duplex, a hairpin, a 90°-kink, a kissing-loop, an open 3-way junction, an open 4-way junction, a stacked 3-way junction, or a 3-way loops, or a combination thereof.

67. The method of claim 36, wherein the electrodes and the substrate are covered by an insulation layer except the end surfaces of the electrodes at the nanogap that are not covered.

68. The method of claim 67, wherein the insulation layer comprises a monolayer or multi-layers of passivated inert chemical.

69. The method of claim 68, wherein the inert chemical comprises 11-mercaptoundecyl-hexaethylene glycol (CR-1) for the metal surface passivation, and aminopropyltriethoxysaline (CR-2) followed by N-hydroxysuccinimidyl 2-(w-O-methoxy-hexaethylene glycol)acetate (CR-3) for the substrate surface passivation.
